(19) United States
(12) Patent Application Publication
(10) Pub. No.: US 2025/0025376 A1
(43) Pub. Date: Jan. 23, 2025

(54) CERAMIC LIFTING AND LOWERING DEVICE FOR THERMOTHERAPEUTIC APPARATUS

(71) Applicant: CERAGEM Co., Ltd., Cheonan-si, Chungcheongnam-do (KR)

(72) Inventors: Dong Myoung LEE, Asan-si, Chungcheongnam-do (KR); Ho Sang YU, Cheonan-si, Chungcheongnam-do (KR); Hyung Joo LEE, Cheonan-si, Chungcheongnam-do (KR)

(21) Appl. No.: 18/714,970

(22) PCT Filed: Nov. 8, 2022
(86) PCT No.: PCT/KR2022/017472

§ 371 Date: May 30, 2024

(30) Foreign Application Priority Data
Dec. 2, 2021 (KR) 10-2021-0170742

(51) Int. Cl. A61H 39/04; A61H 15/00; A61H 15/02; A61H 39/06

(52) U.S. Cl. CPC A61H 39/04; A61H 15/02; A61H 39/06; A61H 2015/0035; A61H 2201/1215; A61H 2201/149; A61H 2201/1676

(57) ABSTRACT

Provided is a ceramic lifting and lowering device for a thermotherapeutic apparatus. The ceramic lifting and lowering device for a thermotherapeutic apparatus according to a first embodiment of the present invention comprises: a base frame; a lifting and lowering member that is hinge-coupled to the base frame; a first driving module provided on the base frame so as to transmit power for the lifting and lowering motion of the lifting and lowering member; a ceramic member provided on one side portion of the lifting and lowering member; a pressing member provided on the other side portion of the lifting and lowering member so as to be movable in an outward direction of the lifting and lowering member; and a second driving module provided on the lifting and lowering member so as to be able to move the pressing member.

### TECHNICAL FIELD

The present invention relates to a ceramic lifting and lowering device for a thermotherapeutic apparatus, and more particularly, to a ceramic lifting and lowering device for a thermotherapeutic apparatus that can massage a specific part of the physical body by pressing it with a predetermined intensity and depth.

### BACKGROUND

Generally, a thermotherapeutic apparatus is in the form of a bed to maximize the effect of heat and far-infrared radiation treatment on the user's spine. This bed-type thermotherapeutic apparatus is equipped with a curved rail similar to the spinal region of the human body in order to provide more accurate and sophisticated treatment effects to the user's spinal region.

A conventional ceramic lifting and lowering device for a thermotherapeutic apparatus is configured to arrange therapeutic ceramics in the form of a janggu (a double-headed drum with a narrow waist in the middle) or a roller that can rotate around a rotation axis, in a plurality of rows. This ceramic lifting and lowering device for a thermotherapeutic apparatus lifts and lowers the therapeutic ceramic according to the user's physical conditions and body shape, and at the same time moves it along the body part to perform thermal treatment.

At this time, the therapeutic ceramic performed a rolling motion in the moving direction and performed acupressure or massage to the user's physical body in order to achieve objectives such as relieving friction between the lifting and lowering device and the physical body due to the movement of the lifting and lowering device and securing the durability of the lifting and lowering device.

However, massage using the rolling motion of such therapeutic ceramics has limitations in the intensity and depth of pressing the physical body.

### SUMMARY

### Technical Tasks

The present invention has been devised in view of the above problems, and is directed to providing a ceramic lifting and lowering device for a thermotherapeutic apparatus capable of performing acupressure on a specific part of the physical body with a desired intensity, direction, and depth.

In addition, the present invention is also directed to providing a ceramic lifting and lowering device for a thermotherapeutic apparatus that can simultaneously provide a massage by rolling motion of a therapeutic ceramic and a massage for performing acupressure on a specific part of the physical body to a desired intensity, direction, and depth.

In addition, the present invention is also directed to providing a ceramic lifting and lowering device for a thermotherapeutic apparatus that can provide the user with a feeling of receiving acupressure by an acupressure rod.

The problems of the present invention are not limited to those mentioned above, and other problems not mentioned will be clearly understood by those of ordinary skill in the art from the following description.

### Technical Solution

According to an aspect of the present invention, provided is a ceramic lifting and lowering device for a thermotherapeutic apparatus, including: a base frame; a lifting and lowering member rotatably coupled to the base frame; a first driving module provided on the base frame to rotate the lifting and lowering member; a ceramic member provided on one side of the lifting and lowering member; a pressing member provided on the other side of the lifting and lowering member to be movable in an outward direction of the lifting and lowering member; and a second driving module provided on the lifting and lowering member to move the pressing member.

In this case, a plurality of the ceramic members may be provided, and each of the plurality of ceramic members may be formed in a cylindrical shape that can rotate around a predetermined rotation axis provided outside the lifting and lowering member with the radius of both sides in the extension direction of the rotation axis being larger than the central portion in the extension direction.

In this case, a plurality of the ceramic members may be provided, and each of the plurality of ceramic members may include a predetermined rotation axis provided outside the lifting and lowering member; and a plurality of rollers rotatably provided on both sides of the rotation axis in the extension direction.

In this case, the plurality of ceramic members may include a first ceramic member provided on one side of the lifting and lowering member, the rotation axis being arranged parallel to the width direction of the lifting and lowering member; a second ceramic member disposed symmetrically with the first ceramic member with respect to the center of the width direction of the lifting and lowering member; a third ceramic member disposed symmetrically with the first ceramic member with respect to the center of the longitudinal direction of the lifting and lowering member; and a fourth ceramic member disposed symmetrically with the second ceramic member with respect to the center of the longitudinal direction of the lifting and lowering member.

In this case, a plurality of the pressing members may be provided, and each of the plurality of pressing members may be disposed adjacent to one of the plurality of ceramic members.

In this case, each of the plurality of pressing members may be located between the plurality of ceramic members.

In this case, the plurality of pressing members may include an inner pressing member disposed adjacent to the central portion of the lifting and lowering member; and an outer pressing member disposed farther from the central portion of the lifting and lowering member than the inner pressing member.

In this case, the inner pressing member may be provided as a pair, and the pair of inner pressing members may be symmetrical with respect to a width direction center line of the lifting and lowering member, and the outer pressing member may be provided as a pair, and the pair of outer pressing members may be symmetrical with respect to the width direction center line of the lifting and lowering member.

In this case, a plurality of the second driving modules may be provided, corresponding to the plurality of pressing members to respectively move the plurality of pressing members.

In this case, the pair of inner pressing members may be provided to move simultaneously at the same height, and the pair of outer pressing members may be provided to move simultaneously at the same height.

In this case, a rack gear may be provided on the lower part of the pressing member parallel to the moving direction of the pressing member, and the second driving module may include a second driving motor; an inner pinion gear rotated by the second driving motor and comprising an arc-shaped gear part engaged with the rack gear of the inner pressing member; and an outer pinion gear rotated by the second driving motor and comprising an arc-shaped gear part engaged with the rack gear of the outer pressing member.

In this case, the second driving module may include a second driving motor; an inner cam part rotated by the second driving motor and in contact with a lower side of the inner pressing member; and an outer cam part rotated by the second driving motor and in contact with a lower side of the outer pressing member, and the inner cam part and the outer cam part may include a protrusion protruding curvedly outward.

In this case, an outer end of the pressing member may be provided to move more outward from the lifting and lowering member in the height direction than the outermost portion of the ceramic member.

In this case, the lifting and lowering member may be provided in a plate shape, and the moving direction of the pressing member may be provided to be perpendicular to one surface of the lifting and lowering member.

In this case, the pressing member may have a rod shape extending in a direction of movement of the pressing member, and an outer end of the pressing member in the moving direction may include a curved surface that is convex in an outward direction.

In this case, the first driving module may include a first driving motor installed on the base frame; a first pinion gear rotated by the first driving motor; and a guide gear provided on the lifting and lowering member to engage with the first pinion gear, and the guide gear may have a curved shape.

In this case, a transfer module for transferring the base frame may be further included.

According to another aspect of the present invention, provided is a ceramic lifting and lowering device for a thermotherapeutic apparatus, including: a base frame; a lifting and lowering member comprising a central part and two side surface parts perpendicular to the central part but facing each other, wherein one side of the two side surface parts is rotatably coupled to the base frame; a first driving module provided on the base frame to rotate the lifting and lowering member; a ceramic member provided on the central part of the lifting and lowering member; a base ceramic member provided on the base frame; and a pressing member positioned between the lifting and lowering ceramic member and the base ceramic member and supported by the base frame.

### Advantageous Effects

According to the embodiments of the present invention, since the pressing member that can move outward of the lifting and lowering member is provided in the lifting and lowering member, it is possible to perform acupressure on a specific part of the physical body with a desired intensity, direction, and depth.

In addition, since the pressing member is located adjacent to the ceramic member that can perform a rolling motion and perform acupressure on the physical body, it is possible to simultaneously provide a massage by rolling motion of a therapeutic ceramic and a massage for performing acupressure on a specific part of the physical body to a desired intensity, direction, and depth.

In addition, the cam part having the protrusion protruding curvedly outward is adjacent at the lower side of the pressing member, and as the cam part rotates, the pressing member moves and presses the user's physical body, whereby it is possible to provide the user with a feeling of receiving acupressure by an acupressure rod.

Advantageous effects of embodiments of the present invention are not limited to the above-described effects, and other effects not mentioned will be clearly understood by those of ordinary skill in the art from the present specification and the accompanying drawings.

### BRIEF DESCRIPTION OF THE DRAWINGS

FIG. 1 is a perspective view of a ceramic lifting and lowering device for a thermotherapeutic apparatus according to a first embodiment of the present invention.

FIG. 2 is a side view of the ceramic lifting and lowering device for a thermotherapeutic apparatus according to the first embodiment of the present invention.

FIGS. 3A and 3B are views illustrating a pressing member, a lifting and lowering member, and a second driving module of the ceramic lifting and lowering device for a thermotherapeutic apparatus according to the first embodiment of the present invention, and FIG. 3A is a front view and FIG. 3B is a side view.

FIG. 4 is a plan view illustrating a lifting and lowering member, a ceramic member, and a pressing member of the ceramic lifting and lowering device for a thermotherapeutic apparatus according to the first embodiment of the present invention.

FIGS. 5A and 5B are views illustrating a state in which the pressing member of the ceramic lifting and lowering device for a thermotherapeutic apparatus according to the first embodiment of the present invention is lowered. For the description of the present invention, only a pressing member, a lifting and lowering member, a ceramic member, and a pinion gear are illustrated.

FIGS. 6A and 6B are views illustrating a state in which an inner pressing member of the ceramic lifting and lowering device for a thermotherapeutic apparatus according to the first embodiment of the present invention is moved downward and an outer pressing member is moved upward. For the description of the present invention, only a pressing member, a lifting and lowering member, a ceramic member, and a pinion gear are illustrated.

FIGS. 7A and 7B are views illustrating a state in which an inner pressing member of the ceramic lifting and lowering device for a thermotherapeutic apparatus according to the first embodiment of the present invention is moved downward and an outer pressing member is moved upward. For the description of the present invention, only a pressing member, a lifting and lowering member, a ceramic member, and a pinion gear are illustrated.

FIG. 8 is a perspective view illustrating a pressing member, a lifting and lowering member, and a second driving module of a ceramic lifting and lowering device for a thermotherapeutic apparatus according to a second embodiment of the present invention.

FIGS. 9A and 9B are views illustrating a pressing member, a lifting and lowering member, and a second driving module of the ceramic lifting and lowering device for a thermotherapeutic apparatus according to the second embodiment of the present invention, and FIG. 9A is a front view and FIG. 9B is a side view.

FIG. 10 is a perspective view of a ceramic lifting and lowering device for a thermotherapeutic apparatus according to a third embodiment of the present invention.

FIGS. 11A and 11B are side views of the ceramic lifting and lowering device for a thermotherapeutic apparatus according to the third embodiment of the present invention, FIG. 11A is a side view illustrating a state in which a lifting and lowering member is lowered, and FIG. 11B is a side view illustrating a state in which the lifting and lowering member is lifted. For the description of the invention, an elastic member is not shown.

### DETAILED DESCRIPTION OF EMBODIMENTS

Hereinafter, embodiments of the present invention will be described in detail so that those of ordinary skill in the art can readily implement the present invention with reference to the accompanying drawings. The present invention may be embodied in many different forms and is not limited to the embodiments set forth herein. In the drawings, parts unrelated to the description are omitted for clarity of description of the present invention, and same or similar reference numerals denote same elements. In addition, the size or shape of components shown in the drawings may be exaggerated for clarity and convenience of explanation.

It is understood that the terms “comprise” or “have” or the like when used in this specification, are intended to specify the presence of stated features, integers, steps, operations, elements, components and/or a combination thereof but not preclude the possibility of the presence or addition of one or more other features, integers, steps, operations, elements, components, or a combination thereof.

In addition, in this specification, spatially relative terms “front”, “rear”, “upper or above or top” or “lower or below or bottom” may be used to describe a correlation with the elements shown in the drawings. These are relative terms determined based on what is shown in the drawings, and the positional relationship may be conversely interpreted according to the orientation.

In addition, the presence of an element in/on “front”, “behind”, “above” or “below” of another element includes not only being disposed in/on “front”, “rear”, “above” or “below” directly in contact with other elements, but also cases in which another element being disposed in the middle, unless otherwise specified.

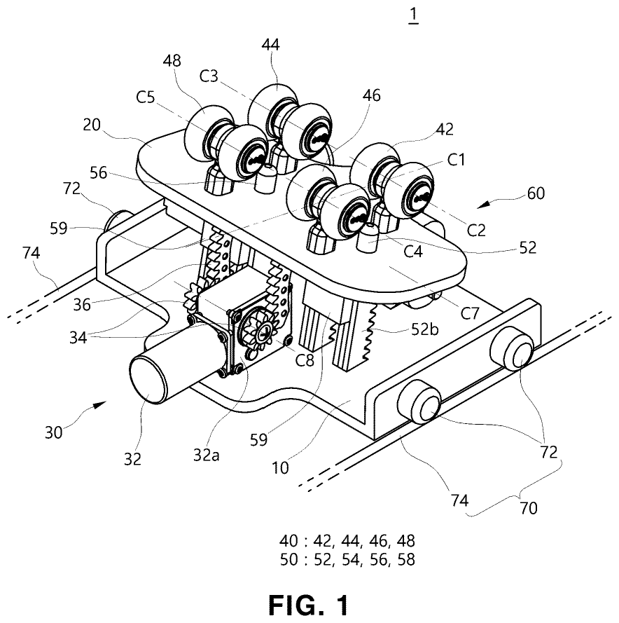

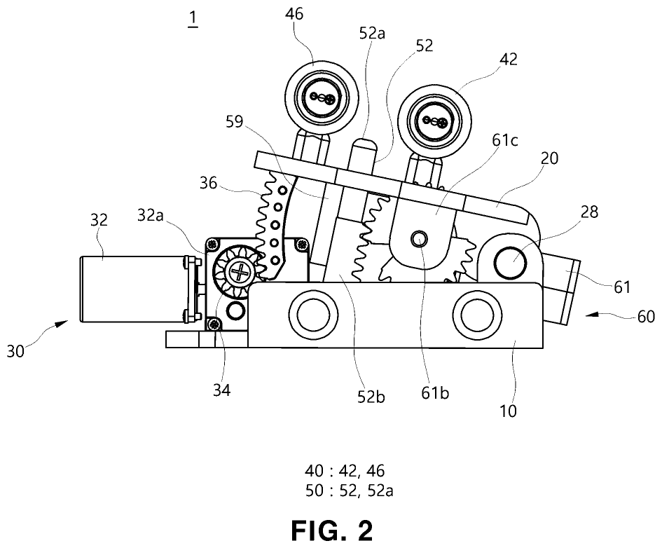

FIG. 1 is a perspective view of a ceramic lifting and lowering device for a thermotherapeutic apparatus according to a first embodiment of the present invention. FIG. 2 is a side view of the ceramic lifting and lowering device for a thermotherapeutic apparatus according to the first embodiment of the present invention.

The ceramic lifting and lowering device for a thermotherapeutic apparatus according to the first embodiment of the present invention is a device capable of pressing a specific part of the physical body with a desired intensity, direction, and depth by including a pressing member as well as a therapeutic ceramic. Accordingly, the ceramic lifting and lowering device for a thermotherapeutic apparatus according to the present embodiment may simultaneously provide a massage by rolling motion of a therapeutic ceramic and a massage for pressing a specific part of the physical body to a desired intensity, direction, and depth.

In this case, the specific part may mean a part where the user wants to receive a massage or an acupuncture point located on the user's physical body.

In this case, an acupuncture point may refer to a part of the physical body that is specified based on traditional Korean medicine. For example, an acupuncture point may refer to a part of the physical body that can relieve the user's fatigue and maintain health during a massage. As a more specific example, an acupuncture point is a meridian related to the bladder, kidneys, brain, heart, etc. in the human body, and may refer to an acupuncture point in the bladder meridian. Meanwhile, the location of the acupuncture point may be different depending on the user's height, weight, gender, age, etc.

Referring to FIGS. 1 and 2, the ceramic lifting and lowering device 1 for a thermotherapeutic apparatus according to the first embodiment of the present invention may include a base frame 10, a lifting and lowering member 20, a first driving module 30, a ceramic member 40, a pressing member 50, a second driving module 60, and a transfer module 70.

The base frame 10 may be a quadrangular plate-shaped member provided so that the lifting and lowering member 20 and the first driving module 30 may be arranged and installed on its upper surface.

The lifting and lowering member 20 may be a plate-shaped member with a size equal to or slightly smaller than that of the base frame 10. The lifting and lowering member 20 may be hinged to a hinge coupling part 28 provided on one side of the base frame 10 to rotate about a hinge axis C7 with respect to the base frame 10.

In this case, the user's physical body may be arranged in a predetermined direction with respect to the lifting and lowering member 20. For example, the user's physical body may be arranged on the upper side of the lifting and lowering member 20 so that the direction from the user's head to the legs is parallel to the longitudinal direction of the lifting and lowering member 20. In this case, the center line of the user's physical body may be arranged to coincide with the width direction center line C1 of the lifting and lowering member 20. Here, the center line of the physical body may mean a straight line connecting the crown of the head and the center of the abdomen, for example, the navel of the abdomen.

Referring again to FIG. 1, the transfer module 70 is installed on one side of the base frame 10, for example, the lower side with respect to FIG. 1, and may transfer the base frame 10 along a specific path. In the present embodiment, the transfer module 70 may be composed of wheels 72 and rails 74.

The wheels 72 may be installed on both sides of the base frame 10. In this case, a plurality of wheels 72 may be installed side by side at a certain distance. The wheel 72 may be seated on the rail 74. In this case, the path of the rail 74 may be provided so that the ceramic member 40 and the pressing member 50 may pass while pressing a specific part of the physical body.

Meanwhile, the moving direction (or the path of the rail 74) of the base frame 10 may be perpendicular to the rotation axes C2, C3, C4, and C5 of the ceramic member 40 to be described later. Accordingly, the ceramic member 40 rotates around the rotation axes C2, C3, C4, and C5 of the ceramic member 40 and may perform a rolling motion in the transfer direction of the transfer module 70.

The ceramic lifting and lowering device 1 for a thermotherapeutic apparatus according to the first embodiment of the present invention may include a driving unit that provides driving force so that the base frame 10 may be transferred along the rail 74. In this case, the driving unit may have various known configurations, such as a motor that rotates the wheel 72 or a power generation apparatus that pushes or pulls one side of the base frame 10 or the like.

Meanwhile, in the present embodiment, a use example in which the center line of the user's physical body is arranged parallel to the upper side of the center line C1 in the width direction of the lifting and lowering member 20, and the base frame 10 is transferred in a direction parallel to the longitudinal direction of the lifting and lowering member 20 will be focused in describing.

The base frame 10 of the ceramic lifting and lowering device 1 for a thermotherapeutic apparatus according to the first embodiment of the present invention may be provided with a first driving module 30 for transmitting power for the lifting and lowering motion of the lifting and lowering member 20.

In the present embodiment, the first driving module 30 may include a first driving motor 32, a first gear box 31a, a first pinion gear 34, and a guide gear 36.

In more detail, the first driving motor 32 may be provided at one side of the base frame 10, for example, at a front portion based on FIG. 1. The first gear box 31a may be provided at one side of the first driving motor 32.

In the present embodiment, a first driving motor rotation shaft may be installed in parallel to the hinge axis C7 inside the first gear box 31a. The driving force generated by the first driving motor 32 may be transmitted to the first driving motor rotation shaft by the first gear box 31a. In this case, the first pinion gear 34 may be installed on the first driving motor rotation shaft.

The guide gear 36 having a curved shape engaged with the first pinion gear 34 may be provided on a lower surface of the lifting and lowering member 20. In this case, the curved shape of the guide gear 36 may have an arc shape with respect to the hinge axis C7 as the center. Accordingly, the lifting and lowering member 20 may perform a lifting and lowering motion while rotating around the hinge axis C7.

In this case, referring to FIGS. 1 and 2, the first driving motor 32 may be installed at the center of the width direction of the base frame 10, and the first pinion gear 34 and the guide gear 36 of the lifting and lowering member 20 may be formed to be symmetrical about the first driving motor 32 on both sides of the first driving motor 32. Accordingly, the lifting and lowering member 20 may be stably supported by the first driving module 30.

Meanwhile, the ceramic member 40 may be formed on the outer surface of the lifting and lowering member 20 to press and massage the user's physical body. In this case, the ceramic member 40 may be made of a thermal ceramic capable of generating heat for thermal treatment.

Meanwhile, each of the plurality of ceramic members 42, 44, 46, and 48 may be formed in a cylindrical shape that can rotate around a predetermined rotation axis C2, C3, C4, C5 installed outside the lifting and lowering member 20 with the radius of both sides in the extension direction of the rotation axis C2, C3, C4, C5 may be formed larger than the central portion in the extension direction. In other words, the ceramic member 40 may have a janggu (a double-headed drum with a narrow waist in the middle) shape.

As a modified example of the ceramic member 40, each of the plurality of ceramic members 42, 44, 46, and 48 may include a predetermined rotation axis C2, C3, C4, C5 installed outside the lifting and lowering member 20, and a plurality of rollers rotatably disposed on both sides in the extension direction of the rotation axis C2, C3, C4, C5.

Accordingly, the plurality of ceramic members 42, 44, 46, and 48 may perform a rolling motion on the user's physical body by rotating around the predetermined rotation axes C2, C3, C4, and C5. In this case, the outermost part of the ceramic member 40 may acupressure or massage the user's physical body.

Here, the outermost part of the ceramic member 40 may mean a part that most protrudes outward from the rotation axis of the ceramic member 40 in the radial direction of the ceramic member 40, and may directly or indirectly contact the user's physical body.

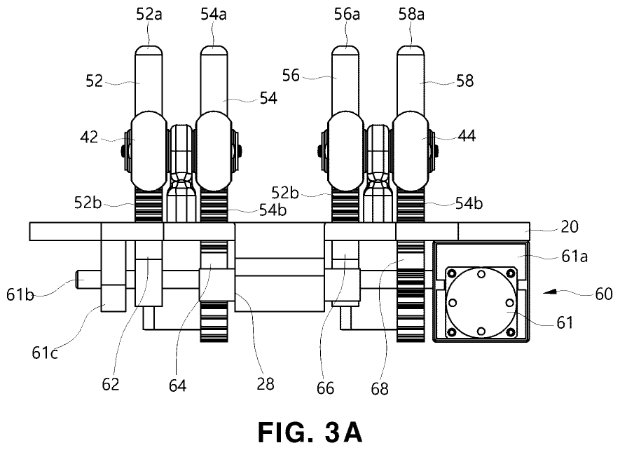

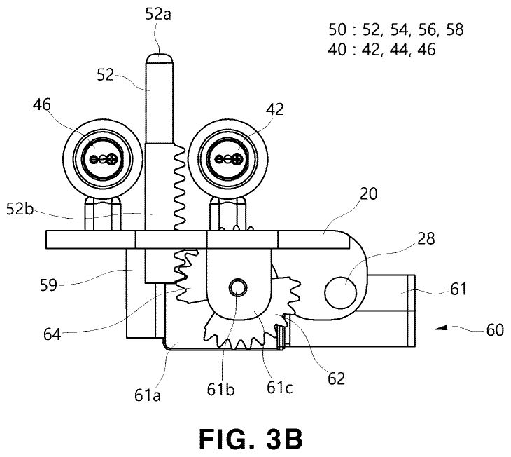

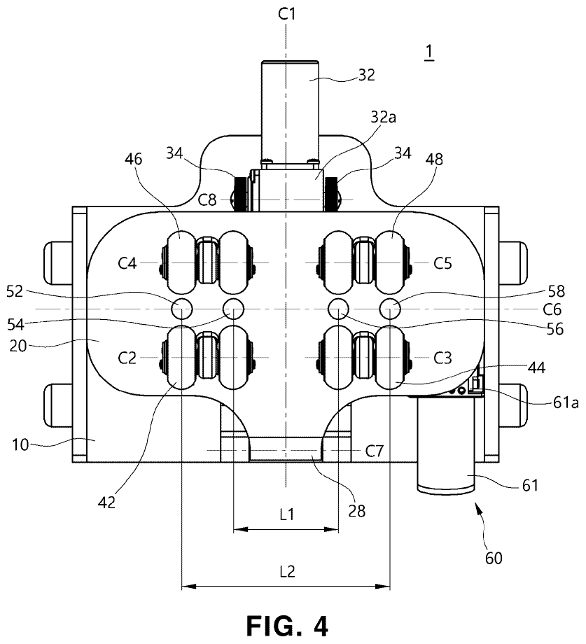

FIGS. 3A and 3B are views illustrating a pressing member, a lifting and lowering member, and a second driving module of the ceramic lifting and lowering device for a thermotherapeutic apparatus according to the first embodiment of the present invention, and FIG. 3A is a front view and FIG. 3B is a side view. FIG. 4 is a plan view illustrating a lifting and lowering member, a ceramic member, and a pressing member of the ceramic lifting and lowering device for a thermotherapeutic apparatus according to the first embodiment of the present invention.

Referring to FIG. 3, a pressing member 50 and a second driving module 60 may be provided on one surface of the lifting and lowering member 20, for example, the lower surface based on FIG. 3B. The second driving module 60 is configured to move the pressing member 50 so as to press or massage the user's physical body.

In this case, the position and pressing direction in which the pressing member 50 presses the user's physical body may be configured to be set by the moving direction of the pressing member 50, and the intensity with which the pressing member 50 presses the user's physical body may be configured to be controlled by the second driving module 60.

In addition, the depth at which the pressing member 50 presses the user's physical body may be determined by at least one of the pressing member 50 and the second driving module 60. Accordingly, the ceramic lifting and lowering device 1 for a thermotherapeutic apparatus according to the present embodiment can pressurize the user's physical body with a desired intensity, direction, and depth.

In the ceramic lifting and lowering device 1 for a thermotherapeutic apparatus according to an embodiment of the present invention, the pressing member 50 may be made of a rod-shaped member extending a predetermined length. In this time, the pressing member 50 may be provided to move in the upward or downward direction of the lifting and lowering member 20. In this case, the pressing member 50 may be arranged so that its longitudinal direction is parallel to the moving direction.

Accordingly, one end of the pressing member 50 can pressurize the physical body, thereby accurately pressing a specific part of the physical body while minimizing the space occupied by the pressing member 50. In this case, a plurality of pressing members 50 may be provided and configured to press several parts of the physical body simultaneously.

Referring to FIGS. 3a, 3b, and 4, in the present embodiment, the plurality of pressing members 50 may include inner pressing members 54 and 56 and outer pressing members 52 and 58. The inner pressing members 54 and 56 may be disposed adjacent to the central portion of the lifting and lowering member 20. The outer pressing members 52 and 58 may be disposed farther from the central portion of the lifting and lowering member 20 than the inner pressing members 54 and 56. Accordingly, the ceramic lifting and lowering device 1 for a thermotherapeutic apparatus according to the present embodiment can simultaneously perform acupressure to parts near and far from the central portion of the user's physical body.

In addition, the inner pressing members 54 and 56 may be formed as a pair symmetrically based on the center line in the width direction of the lifting and lowering member 20. Likewise, the outer pressing members 52 and 58 may be formed as a pair symmetrically based on the center line in the width direction of the lifting and lowering member 20. Accordingly, the pair of inner pressing members 54 and 56 and the pair of outer pressing members 52 and 58 can perform acupressure to symmetrical parts of the physical body's center line.

Meanwhile, the moving direction of the pressing member 50 may be perpendicular to one surface of the lifting and lowering member 20. In this case, since the user's physical body is arranged to be parallel to one surface of the lifting and lowering member 20, the pressing member 50 can press the user's physical body in a direction perpendicular to the user's physical body. Accordingly, the pressing member 50 can strongly press the user's physical body using the driving force generated by the second driving module 60.

Meanwhile, in the present embodiment, the outer end of the pressing member 50 may include a curved surface that is convex outward to more accurately press a specific part of the physical body without causing injury to the user by the end of the pressing member 50.

Referring again to FIGS. 3A and 3B, in the present embodiment, the second driving module 60 may include a second driving motor 61, a second driving motor rotation shaft 61b, a second gear box 61a, and pinion gears 62, 64, 66, and 68.

At the lower part of the pressing member 50, rack gears 52b, 54b, 56b, and 58b may extend in a direction parallel to the moving direction of the pressing member 50.

The second driving motor 61 may be provided on one side of the lifting and lowering member 20, for example, on the rear part with reference to FIG. 3B. The second gear box 61a may be provided at one side of the second driving motor 61. The second driving motor rotation shaft 61b may be provided to penetrate the second gear box 61a. The second gear box 61a may be configured to transmit power generated by the second driving motor 61 to the second driving motor rotation shaft 61b.

Meanwhile, in the present embodiment, a rotation shaft support member 61c capable of supporting the second driving motor rotation shaft 61b may be provided at the other end of the second driving motor rotation shaft 61b. One side of the rotation shaft support member 61c may be fixed to the lifting and lowering member 20, and one side of the second driving motor rotation shaft 61b may be rotatably coupled to the other side. To this end, a bearing (not shown) may be provided on the rotation shaft support member 61c.

The driving force generated by the second driving motor 61 may rotate the second driving motor rotation shaft 61b. As the second driving motor rotation shaft 61b is rotated, the pinion gears 62, 64, 66, and 68 are rotated to move the pressing member 50 to the upper or lower side of the lifting and lowering member 20.

In this case, the pressing members 52, 54, 56, and 58 may move to the inward or outward of the lifting and lowering member 20 by the length of the section in which the rack gears 52b, 54b, 56b, and 58b are formed. Accordingly, by adjusting the length of the section in which the rack gears 52b, 54b, 56b, and 58b are formed, the depth at which the pressing members 52, 54, 56, and 58 press the physical body may be adjusted.

Alternatively, since the length of the section in which the pressing member 50 moves is proportional to the rotation angle of the second driving motor rotation shaft 61b, the depth at which the pressing member 50 presses the physical body may be adjusted by the control of the second driving module 60.

Referring to FIG. 3B, in the present embodiment, the pinion gears 62, 64, 66, and 68 may include arc-shaped gear parts that may be engaged with the rack gears 52b, 54b, 56b, and 58b of the pressing members 52, 54, 56, and 58. Since the rack gears 52b, 54b, 56b, and 58b may move inward or outward of the lifting and lowering member 20 by the arc length of the arc-shaped gear part, the depth at which the pressing member 50 presses the physical body may be adjusted by the shape of the arc-shaped gear part.

Meanwhile, referring to FIGS. 1 and 2 again, in the present embodiment, a guide member 59 protruding downward of the lifting and lowering member 20 may be formed on one surface of the lifting and lowering member 20, for example, on the lower surface based on FIG. 1. In this case, the guide member 59 may extend in a direction parallel to the rack gears 52b, 54b, 56b, and 58b of the pressing members 52, 54, 56, and 58.

Guide protrusions and guide grooves that may be slidably coupled may be formed on the guide member 59 and the rack gears 52b, 54b, 56b, and 58b in parallel with the moving direction of the guide member. For example, the guide member 59 may be provided with a guide protrusion, and a guide groove may be formed at one side of the rack gears 52b, 54b, 56b, and 58b. Accordingly, the guide member 59 may guide the moving direction of the pressing member 50.

Hereinafter, the relative positions of the pressing member and the ceramic member and the corresponding effects will be described. Referring to FIG. 4, in the ceramic lifting and lowering device 1 for a thermotherapeutic apparatus according to the first embodiment of the present invention, a plurality of ceramic members may include a first ceramic member 42 to a fourth ceramic member 48.

The first ceramic member 42 is formed on one side of the lifting and lowering member 20, and may be arranged such that the rotation axis C2 is parallel to the width direction of the lifting and lowering member. The second ceramic member 44 may be provided to be symmetrical to the first ceramic member 42 with respect to the width direction center line C1 of the lifting and lowering member 20.

In addition, the third ceramic member 46 and the fourth ceramic member 48 may be provided to be symmetrical to the first and second ceramic members 42 and 44 based on the longitudinal direction center line C6 of the lifting and lowering member, respectively.

That is, the rotation axes C2, C3, C4, and C5 of the first ceramic member to fourth ceramic member 42, 44, 46, and 48 may be disposed parallel to each other. Accordingly, the plurality of ceramic members 42, 44, 46, and 48 rotate in the same direction and perform rolling motion, thereby performing acupressure or massaging the user's physical body.

Meanwhile, in the present embodiment, each of the plurality of pressing members 52, 54, 56, and 58 may be located adjacent to any one of the plurality of ceramic members 42, 44, 46, and 48. Accordingly, a massage by rolling motion of the ceramic members 42, 44, 46, 48 and a massage by the pressing members 52, 54, 56, 58 that performs acupressure on a specific part of the physical body with a desired intensity, direction and depth can be provided at the same time.

In this case, each of the plurality of pressing members 52, 54, 56, and 58 may be positioned between the plurality of ceramic members 42, 44, 46, and 48 so that the ceramic members 42, 44, 46, and 48 can support most of the user's load. In addition, the plurality of pressing members 52, 54, 56, and 58 may be arranged in a direction parallel to the rotation axes C2, C3, C4, and C5 of the ceramic members 42, 44, 46, and 48.

For example, as shown in FIG. 4, some 52 and 54 of the plurality of pressing members 52, 54, 56, and 58 may be disposed between the first ceramic member 42 and the third ceramic member 46, and the rest 56 and 58 of the plurality of pressing members 52, 54, 56, and 58 may be disposed between the second ceramic member 44 and the fourth ceramic member 48.

Accordingly, a load of the user transmitted to the second driving module 60 through the pressing members 52, 54, 56, and 58 can be reduced. Therefore, a load applied to the second driving module 60 can be reduced and damage to the second driving module 60 can be prevented.

Referring back to FIG. 4, in the present embodiment, each of the plurality of pressing members 52, 54, 56, and 58 may be located between the radial outermost portion of any one of the plurality of ceramic members 42, 44, 46, and 48 and the radial outermost portion of the other ceramic members 42, 44, 46, and 48. In this case, the ceramic member may be a ceramic member adjacent to the longitudinal direction of the lifting and lowering member 20.

Accordingly, when the base frame 10 moves in the longitudinal direction of the lifting and lowering member 20, the ceramic members 42 and 44 rotate and perform a rolling motion on the user's physical body, so that the outermost ends of the ceramic members 42 and 44 can massage the user's physical body.

Thereafter, the pressing members 52, 54, 56, and 58 may perform acupressure on the same position of the physical body in a desired intensity, depth, and direction.

Thereafter, other ceramic members 46, 48 adjacent in the longitudinal direction of the lifting and lowering member 20 perform rolling motion and massage on the same position where acupressure is made by the pressing members 52, 54, 56, and 58, thereby preventing the user's muscles from clumping.

Meanwhile, the distance L1 between the inner pressing members 54 and 56 and the distance L2 between the outer pressing members 52 and 58 may be adjusted depending on a specific part of the physical body to be performed acupressure by the pressing members 52, 54, 56, and 58.

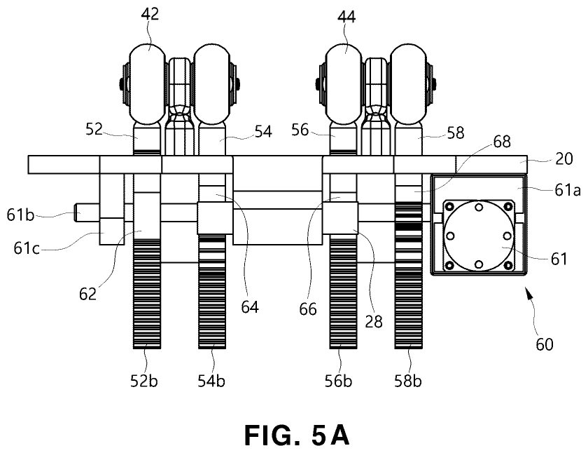

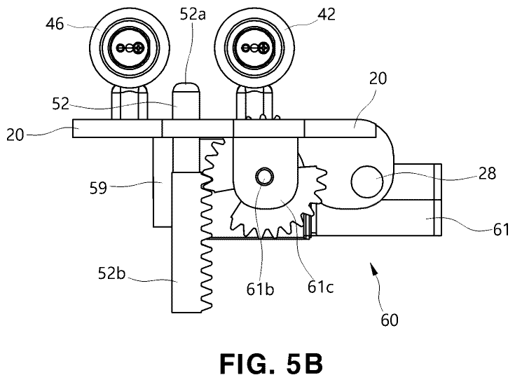

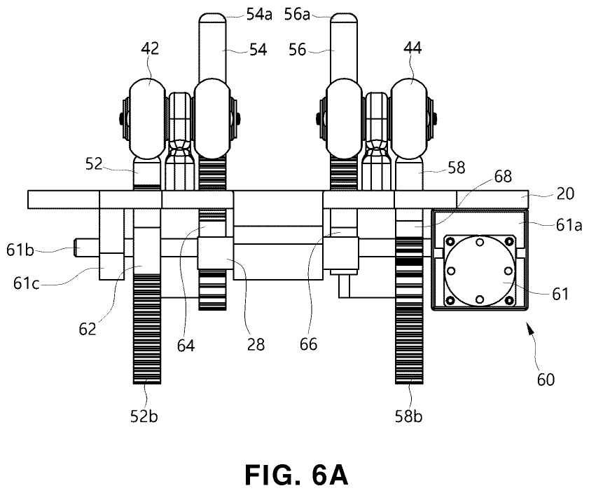

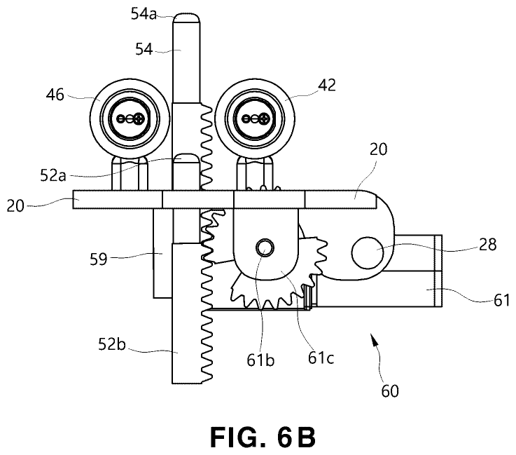

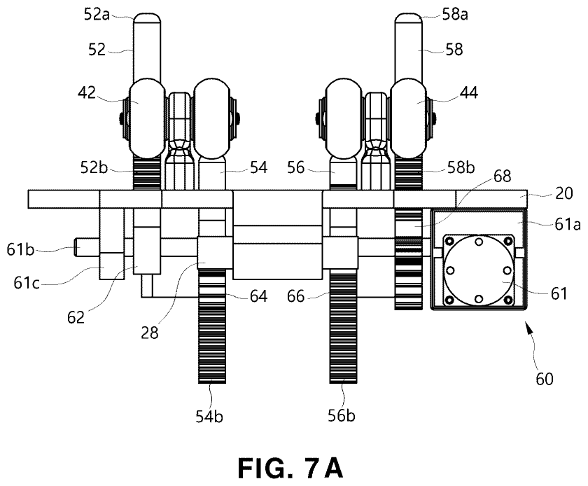

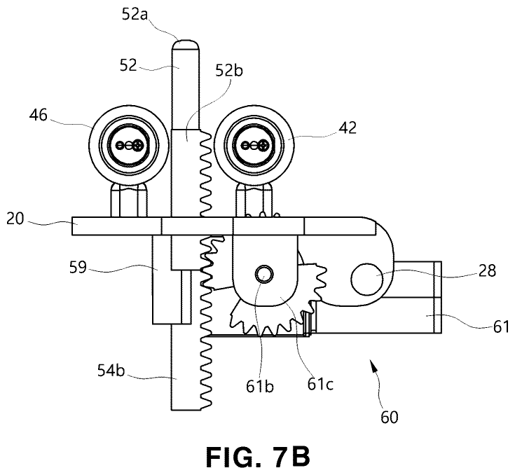

FIGS. 5A and 5B are views illustrating a state in which the pressing member of the ceramic lifting and lowering device for a thermotherapeutic apparatus according to the first embodiment of the present invention is lowered. For the description of the present invention, only a pressing member, a lifting and lowering member, a ceramic member, and a pinion gear are illustrated. FIGS. 6A and 6B are views illustrating a state in which an inner pressing member of the ceramic lifting and lowering device for a thermotherapeutic apparatus according to the first embodiment of the present invention is moved downward and an outer pressing member is moved upward. For the description of the present invention, only a pressing member, a lifting and lowering member, a ceramic member, and a pinion gear are illustrated. FIGS. 7A and 7B are views illustrating a state in which an inner pressing member of the ceramic lifting and lowering device for a thermotherapeutic apparatus according to the first embodiment of the present invention is moved downward and an outer pressing member is moved upward. For the description of the present invention, only a pressing member, a lifting and lowering member, a ceramic member, and a pinion gear are illustrated.

Referring to FIGS. 5A and 5B, in the ceramic lifting and lowering device for a thermotherapeutic apparatus according to the first embodiment of the present invention, the upper ends of the pressing members 52, 54, 56, and 58 may be formed to be movable in the outer direction than the outermost ends of the ceramic members 42, 44, and 46. In this case, the outer direction may mean a direction away from the lifting and lowering member 20.

As the upper ends of the pressing members 52, 54, 56, and 58 move higher outward than the outermost ends of the ceramic members 42, 44, and 46 (that is, as the distance between the upper ends of the pressing members 52, 54, 56, and 58 and the outermost ends of the ceramic members 42, 44, and 46 increases), the load of the physical body supported by the pressing members 52, 54, 56, and 58 increases, so that the user's physical body can be pressed more strongly.

Meanwhile, in the ceramic lifting and lowering device for a thermotherapeutic apparatus according to the first embodiment of the present invention, in order to simultaneously perform acupressure on a position symmetrical with respect to the center line of the physical body to the same depth, a pair of inner pressing members 54 and 56 may be provided to simultaneously move to the same height, and a pair of outer pressing members 52 and 58 may be provided to simultaneously move to the same height.

To this end, the second driving module 60 may include inner pinion gears 64 and 66 for moving the inner pressing members 54 and 56 and outer pinion gears 62 and 68 for moving the outer pressing members 52 and 58. The inner pinion gears 64 and 66 and the outer pinion gears 62 and 68 may be coupled to the same second driving motor rotation shaft 61b.

In this case, the inner pinion gears 64 and 66 may include arc-shaped gear parts that engage with the rack gears 54b and 56b of the inner pressing members 54 and 56, and the outer pinion gears 62 and 68 may include arc-shaped gear parts that engage with the rack gears 52b and 58b of the outer pressing members 52 and 58.

In this case, the gear parts of the inner pinion gears 64 and 66 may be arranged to overlap each other when viewed in the direction of the second driving motor rotation shaft 61b. Likewise, the gear parts of the outer pinion gears 62 and 68 may be arranged to overlap each other when viewed in the direction of the second driving motor rotation shaft 61b.

Accordingly, it is possible to simultaneously move the inner pressing members 54 and 56 and the outer pressing members 52 and 58 to the same height by the shape of the pinion gears 62, 64, 66, and 68 and the rotation of the second driving motor rotation shaft 61b without a separate control device.

In this case, the gear parts of the inner pinion gears 64 and 66 and the gear parts of the outer pinion gears 62 and 68 may be disposed to be misaligned to face different directions when viewed in the direction of the second driving motor rotation shaft 61b. The degree to which both configurations are misaligned may be appropriately adjusted as needed.

Accordingly, it is possible to adjust the acupressure process of the inner pressing members 54 and 56 and the outer pressing members 52 and 58 by adjusting the arrangement of the arc shape of the inner pinion gears 64 and 66 and the arc shape of the outer pinion gears 62, 68 without a separate control device.

Referring to FIGS. 5A and 5B, in the ceramic lifting and lowering device for a thermotherapeutic apparatus according to the first embodiment of the present invention, the arc shape of the inner pinion gears 64 and 66 and the arc shape of the outer pinion gears 62 and 68 may be arranged to face each other with respect to the second driving motor rotation shaft 61b.

In this case, the second driving motor rotation shaft 61b may rotate clockwise based on FIG. 5B so that the arc-shaped gear parts of the inner pinion gears 64 and 66 engage with the rack gears of the pressing members 52, 54, 56, and 58 before the arc-shaped gear parts of the outer pinion gears 62, 68.

Referring to FIGS. 6A and 6B, the inner pinion gears 64 and 66 may engage with the rack gears 54b and 56b of the inner pressing members 54 and 56 and move the inner pressing members 54 and 56 upward. In this case, the rack gears 52b and 58b of the outer pressing members 52 and 58 do not engage with the arc-shaped gear parts of the outer pinion gears 62 and 68, and thus may not be moved outward.

Referring to FIGS. 7A and 7B, since the rack gears 54b and 56b of the inner pressing members 54 and 56 are no longer engaged with the arc-shaped gear parts of the inner pinion gears 64 and 66, they may be moved downward by the load of the inner pressing members 54 and 56 themselves and the load of the physical body. At the same time, the rack gears 52b and 58b of the outer pressing members 52 and 58 may start to engage with the arc-shaped gear parts of the outer pinion gears 62 and 68 and be moved upward.

Referring back to FIGS. 6A and 6B, when the rack gears 54b and 56b of the inner pressing members 54 and 56 engage with arc-shaped gear parts of the inner pinion gears 64 and 66, the inner pressing members 54 and 56 may be moved upward. At the same time, the rack gears 52b and 58b of the outer pressing members 52 and 58 are no longer engaged with the arc-shaped gear parts of the outer pinion gears 62 and 68, so that the outer pressing members 52 and 58 may be moved downward by their own load and physical body load.

As described above, since the up-down movement of the pressing members 52, 54, 56, and 58 is repeatedly performed according to the rotation of the second driving motor rotation shaft 61b, the acupressure process can be controlled so that the massage by the inner pressing members 54 and 56 and the massage by the outer pressing members 52 and 58 are performed sequentially without a separate control device.

In this case, in order to move the pressing members 52, 54, 56, and 58 downward, an elastic member having elastic restoring force may be installed at the lower end of the pressing members 52, 54, 56, and 58. Meanwhile, a plurality of the second driving modules 60 may be formed to correspond to the plurality of pressing members 52, 54, 56, and 58 to move the plurality of pressing members 52, 54, 56, and 58, respectively.

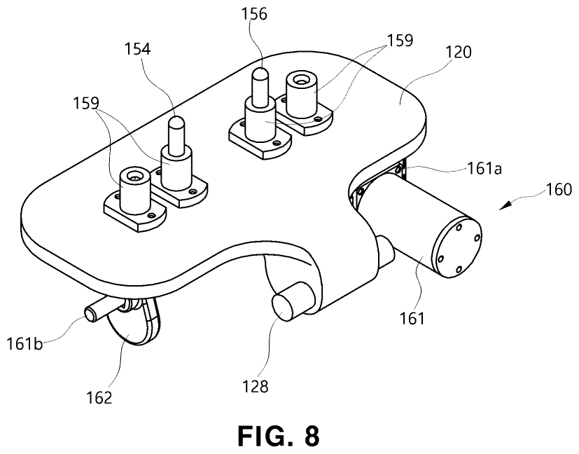

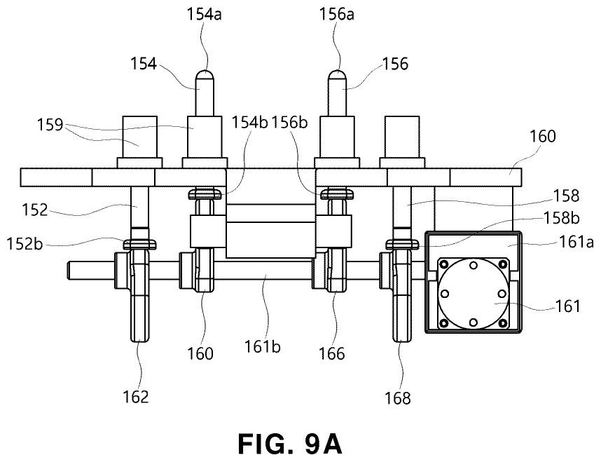

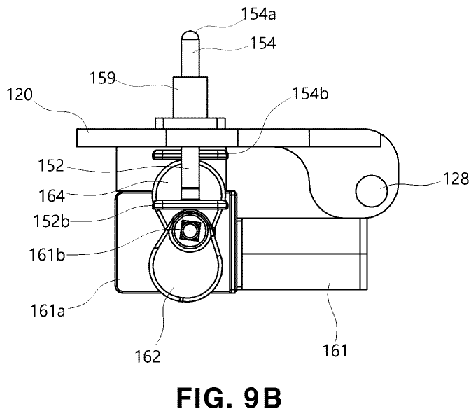

FIG. 8 is a perspective view illustrating a pressing member, a lifting and lowering member, and a second driving module of a ceramic lifting and lowering device for a thermotherapeutic apparatus according to a second embodiment of the present invention. FIGS. 9A and 9B are views illustrating a pressing member, a lifting and lowering member, and a second driving module of the ceramic lifting and lowering device for a thermotherapeutic apparatus according to the second embodiment of the present invention, and FIG. 9A is a front view and FIG. 9B is a side view.

Hereinafter, the ceramic lifting and lowering device for a thermotherapeutic apparatus according to the second embodiment will be described. In the second embodiment of the present invention, the configuration other than the pressing member and the second driving module and the operation method thereof may have the same configuration as in the first embodiment, and thus a description thereof will be omitted, and in the ceramic lifting and lowering device for a thermotherapeutic apparatus according to the second embodiment of the present invention, the second driving module, the pressing member, and the operation thereof will be described in more detail.

Referring to FIGS. 8 and 9, in the ceramic lifting and lowering device for a thermotherapeutic apparatus according to the second embodiment of the present invention, the second driving module 160 may include a second driving motor 161, a second driving motor rotation shaft 161b, a second gear box 161a, and cam parts 162, 164, 166, and 168. In this case, the configuration and operation of the second driving motor 161, the second driving motor rotation shaft 161b, and the second gear box 161a may be the same as those in the first embodiment.

One ends of the pressing members 152, 154, 156, and 158, for example, the lower end based on FIG. 9A, may be provided with driven parts 152b, 154b, 156b, and 158b that are always in contact with the cam parts 162, 164, 166, and 168. A protrusion protruding curvedly outward from the second driving motor rotation shaft 161b may be formed on the outer circumferential surfaces of the cam parts 162, 164, 166, and 168. In this case, the cam parts 162, 164, 166, and 168 may include inner cam parts 164 and 166 and outer cam parts 162 and 168.

The inner cam parts 164 and 166 may be provided to be in contact with the driven parts 154b and 156b of the inner pressing members 154 and 156, and the outer cam parts 162 and 168 may be provided to be in contact with the driven parts 152b and 158b of the outer pressing members 152 and 158.

Meanwhile, a guide member 159 for guiding the moving direction of the pressing members 152, 154, 156, and 158 may be provided on the upper surface of the lifting and lowering member 120. The guide member 159 may protrude upward from the upper surface of the lifting and lowering member 120. A hole through which the pressing members 152, 154, 156, and 158 may move may be provided inside the guide member 159.

The driven parts 152b, 154b, 156b, and 158b of the pressing members 152, 154, 156, and 158 may be always in contact with the outer surfaces of the curved cam parts 162, 164, 166, and 168, and as the cam parts 162, 164, 166, and 168 rotate, the pressing members 152, 154, 156, and 158 may move to press the user's physical body. Accordingly, the ceramic lifting and lowering device for a thermotherapeutic apparatus according to the second embodiment of the present invention can provide the user with a feeling of receiving acupressure by an acupressure rod.

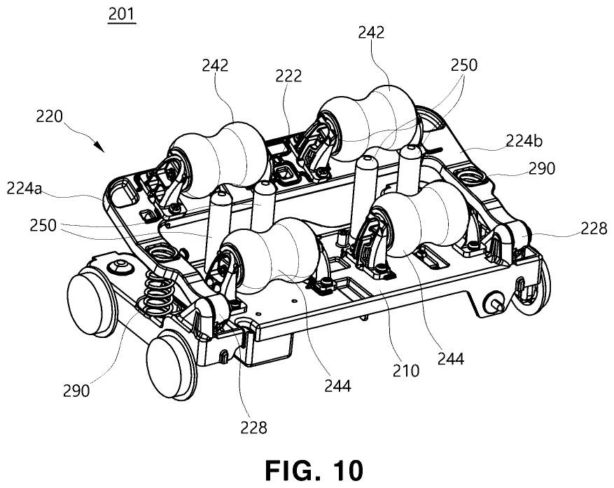

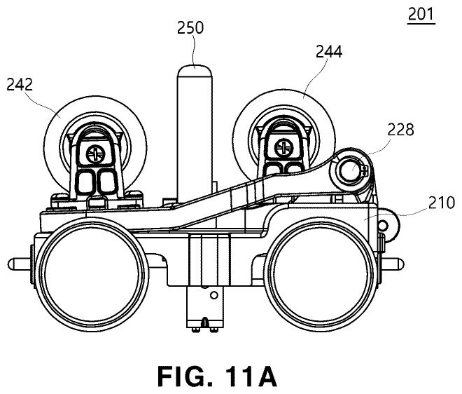

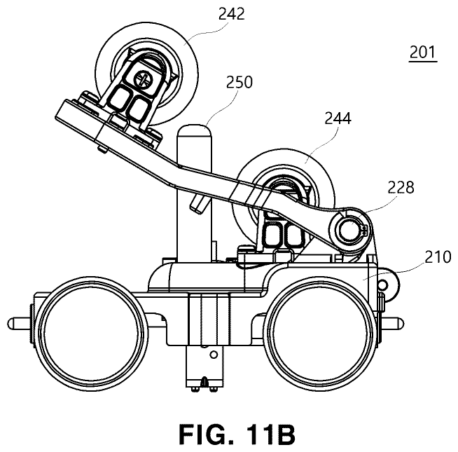

FIG. 10 is a perspective view of a ceramic lifting and lowering device for a thermotherapeutic apparatus according to a third embodiment of the present invention. FIGS. 11A and 11B are side views of the ceramic lifting and lowering device for a thermotherapeutic apparatus according to the third embodiment of the present invention, FIG. 11A is a side view illustrating a state in which a lifting and lowering member is lowered, and FIG. 11B is a side view illustrating a state in which the lifting and lowering member is lifted. For the description of the invention, an elastic member is not shown.

Hereinafter, the ceramic lifting and lowering device for a thermotherapeutic apparatus according to the third embodiment will be described. Since a first driving module of the ceramic lifting and lowering device for a thermotherapeutic apparatus according to the third embodiment and the operation method thereof may have the same configuration as in the first embodiment, a detailed description thereof will be omitted, and a lifting and lowering member, an arrangement of a ceramic member, and a pressing member of the ceramic lifting and lowering device for a thermotherapeutic apparatus according to the third embodiment of the present invention will be described in more detail.

Referring to FIG. 10, in the ceramic lifting and lowering device 201 for a thermotherapeutic apparatus according to the third embodiment of the present invention, the lifting and lowering member 220 may include a central part 222 and two side surface parts 224a and 224b.

The central part 222 may be formed of a frame extending in a direction parallel to the width direction of the base frame 210. The two side surface parts 224a and 224b may extend vertically from the central part 222 by a predetermined length and may be formed of rod-shaped frames facing each other.

One end of the both side parts 224a and 224b of the lifting and lowering member 220, for example, a lower end part based on FIG. 10, may be hinge-coupled to the base frame 210 by a hinge coupling part 228 so that the lifting and lowering member 220 may rotate with respect to the base frame 210.

A lifting and lowering ceramic member 242 may be provided in the central part 222 of the lifting and lowering member 220. A base ceramic member 244 may be provided on the upper surface of the base frame 210. In this case, the base ceramic member 244 may be disposed between the two side surface parts 224a and 224b of the lifting and lowering member 220. An elastic member 290 may be provided on the two side surface parts 224a and 224b of the lifting and lowering member 220 to assist the lifting and lowering motion of the lifting and lowering member 220.

In the present embodiment, a pressing member 250 protruding outward from the base frame 210 may be provided on the base frame 210. In this case, the pressing member 250 may be positioned between the base ceramic member 244 and the lifting and lowering ceramic member 242. One side of the pressing member 250, for example, the lower side based on FIG. 10, may be fixed to the base frame 210. That is, the pressing member 250 may be supported by the base frame 210.

Accordingly, according to the present embodiment, a massage by rolling motion of the ceramic members 242 and 244 and a massage by the pressing member 250 that performs acupressure on a specific part of the physical body with a desired intensity, direction and depth can be provided at the same time.

In addition, according to the present embodiment, since the pressing member 250 supports the user's load, it is possible to reduce the user's load transmitted to the first driving module 230 through the lifting and lowering ceramic member 242. Furthermore, since the reaction force to the user's load supported by the pressing member 250 is applied to the user's physical body, the pressing member 250 can massage the user's physical body with strong intensity.

Accordingly, since the ceramic lifting and lowering device 201 for a therapeutic apparatus according to the third embodiment of the present invention may not include a separate driving module for moving the pressing member 250, the structure of the lifting and lowering member 220 can be simplified.

In addition, when the pressing member 250 presses the user's physical body, the user's load supported by the pressing member 250 is directly supported by the base frame 210, so that the user's physical body can be massaged with stronger intensity.

Meanwhile, in the ceramic lifting and lowering device 201 for a thermotherapeutic apparatus according to the third embodiment of the present invention, the height at which the pressing member 250 protrudes to the outside of the base frame 210 needs to be adjusted so that the pressing member 250 presses a specific part of the physical body when the pressing member 250 is moved upward and does not press the physical body when the pressing member 250 is moved downward.

Referring to FIG. 11A, in a state in which the lifting and lowering member 220 is lowered, the upper end of the pressing member 250 may be positioned to protrude further outward from the base frame 210 than the outermost end of the ceramic members 242 and 244. Accordingly, most of the user's load can be supported by the pressing member 250, and the pressing member 250 can strongly massage the user's physical body.

Referring to FIG. 11B, in a state in which the lifting and lowering member 220 is lifted, the outermost end of the lifting and lowering ceramic member 242 of the lifting and lowering member 220 may be positioned to protrude further outward from the base frame 210 than the upper end of the pressing member 250. Accordingly, the user's load is supported by the lifting and lowering ceramic member 242 and the base ceramic member 244, and the pressing member 250 may not press the user's physical body.

As previously discussed, since the ceramic lifting and lowering device for a thermotherapeutic apparatus according to the first embodiment of the present invention has a pressing member that can move outward of the lifting and lowering member in the lifting and lowering member, it is possible to perform acupressure on a specific part of the physical body with a desired intensity, direction, and depth.

In addition, the ceramic lifting and lowering device for a thermotherapeutic apparatus according to the first embodiment of the present invention has the pressing member located adjacent to the ceramic member that can perform a rolling motion and perform acupressure on the physical body, and thus can simultaneously provide a massage by rolling motion of a therapeutic ceramic and a massage for performing acupressure on a specific part of the physical body to a desired intensity, direction, and depth.

In addition, in the ceramic lifting and lowering device for a thermotherapeutic apparatus according to the second embodiment of the present invention, the cam part having the protrusion protruding curvedly outward is provided adjacent at the lower side of the pressing member, and as the cam part rotates, the pressing member moves and presses the user's physical body, whereby it can provide the user with a feeling of receiving acupressure by an acupressure rod.

In addition, the ceramic lifting and lowering device for a thermotherapeutic apparatus according to the third embodiment of the present invention can massage the user's physical body with strong intensity since it is configured that the user's load supported by the pressing member is to be supported by the base frame.

Although embodiments of the present invention have been described above, the idea of the present invention is not limited to the embodiments set forth herein. Those of ordinary skill in the art who understand the idea of the present invention may easily propose other embodiments through supplement, change, removal, addition, etc. of elements within the scope of the same idea, but the embodiments will be also within the idea scope of the present invention.

1. A ceramic lifting and lowering device for a thermotherapeutic apparatus, comprising:
a base frame;
a lifting and lowering member rotatably coupled to the base frame;
a first driving module provided on the base frame to rotate the lifting and lowering member;
a ceramic member provided on one side of the lifting and lowering member;
a pressing member provided on the other side of the lifting and lowering member to be movable in an outward direction of the lifting and lowering member; and
a second driving module provided on the lifting and lowering member to move the pressing member.

2. The ceramic lifting and lowering device for a thermotherapeutic apparatus of claim 1,
wherein a plurality of the ceramic members are provided, and
wherein each of the plurality of ceramic members is formed in a cylindrical shape that can rotate around a predetermined rotation axis provided outside the lifting and lowering member with the radius of both sides in the extension direction of the rotation axis being larger than the central portion in the extension direction.

3. The ceramic lifting and lowering device for a thermotherapeutic apparatus of claim 1,
wherein a plurality of the ceramic members are provided, and
wherein each of the plurality of ceramic members comprises:
a predetermined rotation axis provided outside the lifting and lowering member; and
a plurality of rollers rotatably provided on both sides of the rotation axis in the extension direction.

4. The ceramic lifting and lowering device for a thermotherapeutic apparatus of claim 2,
wherein the plurality of ceramic members comprises:
a first ceramic member provided on one side of the lifting and lowering member, the rotation axis being arranged parallel to the width direction of the lifting and lowering member;
a second ceramic member disposed symmetrically with the first ceramic member with respect to the center of the width direction of the lifting and lowering member;
a third ceramic member disposed symmetrically with the first ceramic member with respect to the center of the longitudinal direction of the lifting and lowering member; and
a fourth ceramic member disposed symmetrically with the second ceramic member with respect to the center of the longitudinal direction of the lifting and lowering member.

5. The ceramic lifting and lowering device for a thermotherapeutic apparatus of claim 4,
wherein a plurality of the pressing members are provided, and
wherein each of the plurality of pressing members is disposed adjacent to one of the plurality of ceramic members.

6. The ceramic lifting and lowering device for a thermotherapeutic apparatus of claim 5, wherein each of the plurality of pressing members is located between the plurality of ceramic members.

7. The ceramic lifting and lowering device for a thermotherapeutic apparatus of claim 6,
wherein the plurality of pressing members comprises:
an inner pressing member disposed adjacent to the central portion of the lifting and lowering member; and
an outer pressing member disposed farther from the central portion of the lifting and lowering member than the inner pressing member.

8. The ceramic lifting and lowering device for a thermotherapeutic apparatus of claim 7,
wherein the inner pressing member is provided as a pair, and
the pair of inner pressing members are symmetrical with respect to a width direction center line of the lifting and lowering member, and
wherein the outer pressing member is provided as a pair, and
the pair of outer pressing members are symmetrical with respect to the width direction center line of the lifting and lowering member.

9. The ceramic lifting and lowering device for a thermotherapeutic apparatus of claim 8, wherein a plurality of the second driving modules are provided, corresponding to the plurality of pressing members to respectively move the plurality of pressing members.

10. The ceramic lifting and lowering device for a thermotherapeutic apparatus of claim 8,
wherein the pair of inner pressing members are provided to move simultaneously at the same height, and
wherein the pair of outer pressing members are provided to move simultaneously at the same height.

11. The ceramic lifting and lowering device for a thermotherapeutic apparatus of claim 10,
wherein a rack gear is provided on the lower part of the pressing member parallel to the moving direction of the pressing member, and
wherein the second driving module comprises:
a second driving motor;
an inner pinion gear rotated by the second driving motor and comprising an arc-shaped gear part engaged with the rack gear of the inner pressing member; and
an outer pinion gear rotated by the second driving motor and comprising an arc-shaped gear part engaged with the rack gear of the outer pressing member.

12. The ceramic lifting and lowering device for a thermotherapeutic apparatus of claim 10,
wherein the second driving module comprises:
a second driving motor;
an inner cam part rotated by the second driving motor and in contact with a lower side of the inner pressing member; and
an outer cam part rotated by the second driving motor and in contact with a lower side of the outer pressing member, and
wherein the inner cam part and the outer cam part comprise a protrusion protruding curvedly outward.

13. The ceramic lifting and lowering device for a thermotherapeutic apparatus of claim 1, wherein an outer end of the pressing member is provided to move more outward from the lifting and lowering member in the height direction than the outermost portion of the ceramic member.

14. The ceramic lifting and lowering device for a thermotherapeutic apparatus of claim 1,
wherein the lifting and lowering member is provided in a plate shape, and
wherein the moving direction of the pressing member is provided to be perpendicular to one surface of the lifting and lowering member.

15. The ceramic lifting and lowering device for a thermotherapeutic apparatus of claim 1, wherein the pressing member has a rod shape extending in a direction of movement of the pressing member, and an outer end of the pressing member in the moving direction comprises a curved surface that is convex in an outward direction.

16. The ceramic lifting and lowering device for a thermotherapeutic apparatus of claim 1,
wherein the first driving module comprises:
a first driving motor installed on the base frame;
a first pinion gear rotated by the first driving motor; and
a guide gear provided on the lifting and lowering member to engage with the first pinion gear, and
wherein the guide gear has a curved shape.

17. The ceramic lifting and lowering device for a thermotherapeutic apparatus of claim 1, further comprising a transfer module for transferring the base frame.

18. A ceramic lifting and lowering device for a thermotherapeutic apparatus, comprising:
a base frame;
a lifting and lowering member comprising a central part and two side surface parts perpendicular to the central part but facing each other, wherein one side of the two side surface parts is rotatably coupled to the base frame;
a first driving module provided on the base frame to rotate the lifting and lowering member;
a ceramic member provided on the central part of the lifting and lowering member;
a base ceramic member provided on the base frame; and
a pressing member positioned between the lifting and lowering ceramic member and the base ceramic member and supported by the base frame.
